(19) United States
(12) Patent Application Publication
(10) Pub. No.: US 2022/0362560 A1
(43) Pub. Date: Nov. 17, 2022

(54) SYSTEMS AND METHODS FOR MEASURING TEMPERATURE ON OR NEAR AN IMPLANTABLE MEDICAL DEVICE

(71) Applicant: Boston Scientific Neuromodulation Corporation, Valencia, CA (US)

(72) Inventor: Emanuel Feldman, Simi Valley, CA (US)

(21) Appl. No.: 17/771,775

(22) PCT Filed: Oct. 26, 2020
(86) PCT No.: PCT/US20/57353

§ 371 Date: Apr. 25, 2022

Related U.S. Application Data
(60) Provisional application No. 62/927,024, filed on Oct. 28, 2019.

(51) Int. Cl. A61N 1/375; A61N 1/378; A61N 1/05

(52) U.S. Cl. CPC A61N 1/3758; A61N 1/378; A61N 1/05

(57) ABSTRACT

An implantable medical device, such as an implantable pulse generator, includes a case; an integrated circuit device disposed within the case, the integrated circuit device including a temperature sensor; and a thermal coupling medium disposed between, and in contact with, the case and the integrated circuit device, wherein the thermal coupling medium is a solid, liquid, gel, or any combination thereof.

### CROSS-REFERENCE TO RELATED APPLICATIONS

This application claims the benefit under 35 U.S.C. § 119(e) of U.S. Provisional Patent Application Ser. No. 62/927,024, filed Oct. 28, 2019, which is incorporated herein by reference.

### FIELD

The present invention is directed to the area of implantable stimulation systems and methods of making and using the systems. The present invention is also directed to systems and methods for temperature measurement on or near a case of an implantable medical device.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include an implantable pulse generator, one or more leads, and an array of stimulator electrodes on each lead. The stimulator electrodes are in contact with or near the nerves, muscles, or other tissue to be stimulated. The implantable pulse generator generates electrical pulses that are delivered by the electrodes to body tissue.

### BRIEF SUMMARY

One aspect is an implantable medical device, such as an implantable pulse generator, that includes a case; an integrated circuit device disposed within the case, the integrated circuit device including a temperature sensor; and a thermal coupling medium disposed between, and in contact with, the case and the integrated circuit device, wherein the thermal coupling medium is a solid, liquid, gel, or any combination thereof.

In at least some aspects, the thermal coupling medium includes a thermal adhesive, thermal tape, thermal paste, thermal grease, thermal pad, or any combination thereof. In at least some aspects, the thermal coupling medium includes a thermal epoxy. In at least some aspects, the thermal coupling medium has a thermal conductivity that is at least five times a thermal conductivity of air. In at least some aspects, the thermal coupling medium is not electrically conductive.

In at least some aspects, the implantable medical device further includes a printed circuit board upon which the integrated circuit device is mounted. In at least some aspects, the integrated circuit device is disposed between the printed circuit board and the thermal coupling medium. In at least some aspects, the implantable medical device further includes a power source disposed within the case.

In at least some aspects, the integrated circuit device includes a processor configured for controlling operation of the implantable medical device. In at least some aspects, the implantable medical device further includes a memory coupled to the processor. In at least some aspects, the memory includes instructions, wherein the processor is configured to execute the instructions to perform actions including measuring a temperature using the temperature sensor. In at least some aspects, the actions further include determining whether the implantable medical device has been implanted based on the measured temperature. In at least some aspects, the actions further include recording the measured temperature in the memory. In at least some aspects, the actions further include sending a warning when the measured temperature exceeds a threshold value. In at least some aspects, the actions further include halting operation of the implantable medical device when the measured temperature exceeds a threshold value.

Another aspect is an electrical stimulation system that includes any of the implantable medical device described above; and a stimulation lead configured for implantation into a patient and coupleable to the implantable medical device, the stimulation lead including a lead body having a distal end portion and a proximal end portion, electrodes disposed at the distal end portion of the lead body, terminals disposed at the proximal end portion of the lead body, and conductive wires coupling the electrodes electrically to the terminals.

In at least some aspects, the electrical stimulation system further includes a lead extension coupleable between the implantable medical device and the stimulation lead. In at least some aspects, the thermal coupling medium includes a thermal adhesive, thermal tape, thermal paste, thermal grease, thermal pad, or any combination thereof.

A further aspect is a method of measuring a temperature of, or near, a case of an implantable medical device. The method includes providing any of the implantable medical device described above and measuring a temperature using the temperature sensor. In at least some aspects, the method further includes sending a warning or halting operation of the implantable medical device if the measured temperature exceeds a threshold value.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of another embodiment of an electrical stimulation system that includes a percutaneous lead body coupled to an implantable pulse generator;

FIG. 2A is a schematic view of one embodiment of a plurality of connector assemblies disposed in the implantable pulse generator of FIG. 1, the connector assemblies configured to receive the proximal portions of the lead bodies of FIG. 1;

FIG. 2B is a schematic view of one embodiment of a proximal portion of the lead body of FIG. 1, a lead extension, and the implantable pulse generator of FIG. 1, the lead extension configured to couple the lead body to the implantable pulse generator;

FIG. 3 is a schematic overview of one embodiment of components of a stimulation system, including an electronic subassembly disposed within an implantable pulse generator, according to the invention;

FIG. 4 is a schematic cross-sectional view of another embodiment of an implantable pulse generator (or other implantable medical device);

FIG. 5 is a schematic cross-sectional view of a portion of a further embodiment of an implantable pulse generator (or other implantable medical device); and

FIG. 6 is a flow chart of one embodiment of a method of measuring a temperature of, or near, a case of an implantable pulse generator or other implantable medical device.

### DETAILED DESCRIPTION

The present invention is directed to the area of implantable stimulation systems and methods of making and using the systems. The present invention is also directed to systems and methods for temperature measurement on or near a case of an implantable medical device, such as an implantable pulse generator. The present invention can be utilized with any suitable implantable medical device. An implantable pulse generator is utilized as an example of an implantable medical device for illustration purposes, but it will be understood that the invention is applicable to other implantable medical devices.

Suitable implantable electrical stimulation systems include, but are not limited to, an implantable pulse generator and a least one lead with one or more electrodes disposed along a distal end of the lead and one or more terminals disposed along the one or more proximal ends of the lead. Examples of electrical stimulation systems with leads, which can be modified as described herein to facilitate temperature measurement, are found in, for example, U.S. Pat. Nos. 6,181,969; 6,295,944; 6,391,985; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,836,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0135817; 2011/0135818; 2011/0238129; 2011/0363500; 2012/0016378; 2012/0046710; 2012/0071949; 2012/0165911; 2012/0197375; 2012/0203366; 2012/0203320; 2012/0203321; 2012/0366615; 2013/0105071; 2011/0005069; 2010/0268298; 2011/0135817; 2011/0135818; 2011/0078900; 2011/0238129; 2011/0363500; 2012/0016378; 2012/0046710; 2012/0165911; 2012/0197375; 2012/0203366; 2012/0203320; and 2012/0203321, all of which are incorporated by reference in their entireties.

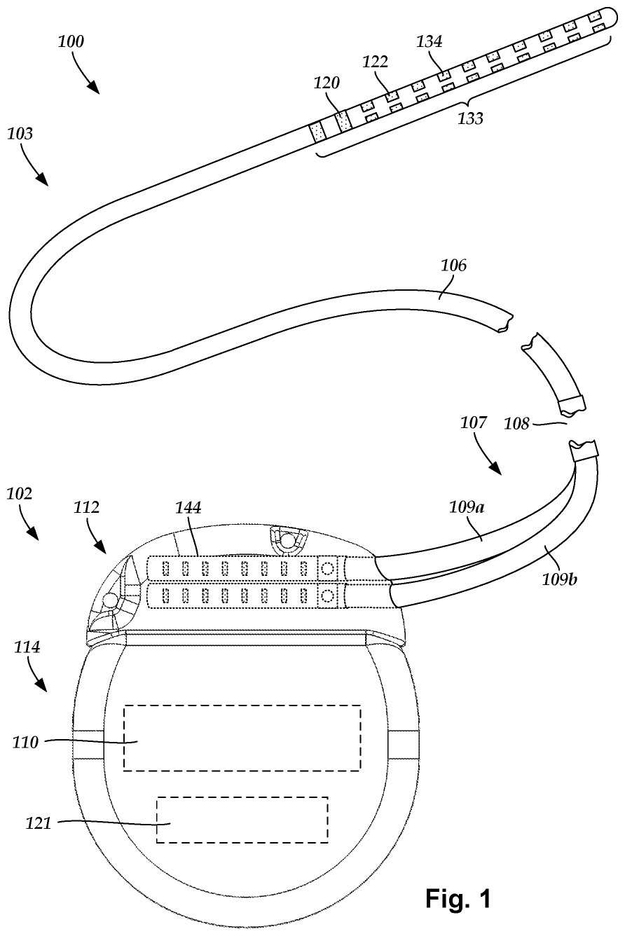

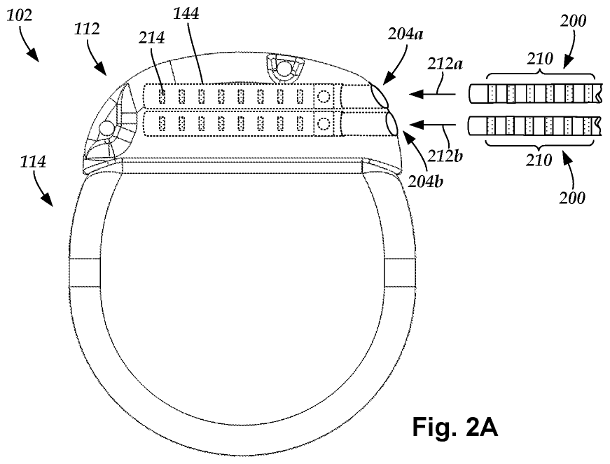

FIG. 1 illustrates schematically one embodiment of an electrical stimulation system 100. The electrical stimulation system includes an implantable pulse generator (IPG) 102 and at least one lead 103 coupleable to the implantable pulse generator 102. The lead 103 includes one or more lead bodies 106, an array of electrodes 133, such as electrode 134, and an array of terminals (e.g., 210 in FIGS. 2A and 2B) disposed along the one or more lead bodies 106. In at least some embodiments, the lead is isodiametric along a longitudinal length of the lead body 106. FIG. 1 illustrates one lead 103 coupled to an implantable pulse generator 102. Other embodiments may include two, three, four, or more leads 103 coupled to the implantable pulse generator 102.

The lead 103 can be coupled to the implantable pulse generator 102 in any suitable manner. In at least some embodiments, the lead 103 couples directly to the implantable pulse generator 102. In at least some other embodiments, the lead 103 couples to the implantable pulse generator 102 via one or more intermediate devices. For example, in at least some embodiments one or more lead extensions 224 (see e.g., FIG. 2B) can be disposed between the lead 103 and the implantable pulse generator 102 to extend the distance between the lead 103 and the implantable pulse generator 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the implantable pulse generator 102, the intermediate devices may be configured into any suitable arrangement.

In FIG. 1, the electrical stimulation system 100 is shown having a splitter 107 configured and arranged for facilitating coupling of the lead 103 to the implantable pulse generator 102. The splitter 107 includes a splitter connector 108 configured to couple to a proximal end of the lead 103, and one or more splitter tails 109a and 109b configured and arranged to couple to the implantable pulse generator 102 (or another splitter, a lead extension, an adaptor, or the like).

The implantable pulse generator 102 can include a connector housing 112 and an IPG case 114. In at least some embodiments, the connector housing 112 can be part of the IPG case 114. An electronic subassembly 110 and an optional power source 121 are disposed in a portion of the IPG case 114 that is preferentially sealed (more preferentially, hermetically sealed). At least one IPG connector 144 is disposed in the connector housing 112. The IPG connector 144 is configured and arranged to make an electrical connection between the lead 103 and the electronic subassembly 110 of the implantable pulse generator 102.

The electrical stimulation system or components of the electrical stimulation system, including one or more of the lead bodies 106 and the implantable pulse generator 102, are typically implanted into the body of a patient. The electrical stimulation system can be used for a variety of applications including, but not limited to, brain stimulation, neural stimulation, spinal cord stimulation, muscle stimulation, peripheral nerve stimulation, or the like.

The electrodes 134 of the lead 103 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of the one or more lead bodies 106 are typically disposed in, or separated by, a non-conductive, biocompatible material such as, for example, silicone, polyurethane, polyetheretherketone (“PEEK”), epoxy, and the like or combinations thereof. The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

Terminals (e.g., 210 in FIGS. 2A and 2B) are typically disposed along the proximal end of the one or more lead bodies 106 of the electrical stimulation system 100 (as well as any splitters, lead extensions, adaptors, or the like) for electrical connection to corresponding connector contacts (e.g., 214 in FIG. 2A and 240 in FIG. 2B). The connector contacts are disposed in connectors (e.g., 144 in FIGS. 1-2B; and 221 in FIG. 2B) which, in turn, are disposed on, for example, the implantable pulse generator 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

FIG. 2A is a schematic side view of one embodiment of a proximal end of one or more elongated devices 200 configured and arranged for coupling to one embodiment of the IPG connector 144. The one or more elongated devices may include, for example, the lead body 106, one or more intermediate devices (e.g., the splitter 107 of FIG. 1, the lead extension 224 of FIG. 2B, an adaptor, or the like or combinations thereof), or a combination thereof. FIG. 2A illustrates two elongated devices 200 coupled to the implantable pulse generator 102. These two elongated devices 200 can be two tails as illustrated in FIG. 1 or two different leads or any other combination of elongated devices.

The IPG connector 144 defines at least one port into which a proximal end of the elongated device 200 can be inserted, as shown by directional arrows 212a and 212b. In FIG. 2A (and in other figures), the connector housing 112 is shown having two ports 204a and 204b. The connector housing 112 can define any suitable number of ports including, for example, one, two, three, four, five, six, seven, eight, or more ports.

The IPG connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the implantable pulse generator 102 to the electrodes (134 of FIG. 1) disposed at a distal end of the lead 103. Examples of connectors in implantable pulse generators are found in, for example, U.S. Pat. Nos. 7,244,150 and 8,224,450, which are incorporated by reference in their entireties.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, the splitter 107, an adaptor, another lead extension, or the like or combinations thereof) to the implantable pulse generator 102. In FIG. 2B, the lead extension 224 is shown coupled to a single port 204 defined in the IPG connector 144. Additionally, the lead extension 224 is shown configured and arranged to couple to a single elongated device 200. In alternate embodiments, the lead extension 224 is configured and arranged to couple to multiple ports 204 defined in the IPG connector 144, or to receive multiple elongated devices 200, or both.

A lead extension connector 221 is disposed on the lead extension 224. In FIG. 2B, the lead extension connector 221 is shown disposed at a distal end 226 of the lead extension 224. The lead extension connector 221 includes a connector housing 228. The connector housing 228 defines at least one port 235 into which terminals 210 of the elongated device 200 can be inserted, as shown by directional arrow 238. The connector housing 228 also includes a plurality of connector contacts, such as connector contact 240. When the elongated device 200 is inserted into the port 235, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the IPG connector 144.

In FIG. 1, the electrodes 134 are shown as including both ring electrodes 120 and segmented electrodes 122. In some embodiments, the electrodes 134 are all segmented electrodes or all ring electrodes. The segmented electrodes 122 of FIG. 1 are in sets of three (one of which is not visible in FIG. 1), where the three segmented electrodes of a particular set are electrically isolated from one another and are circumferentially offset along the lead 1-3. Any suitable number of segmented electrodes can be formed into a set including, for example, two, three, four, or more segmented electrodes. The lead 103 of FIG. 1 has thirty segmented electrodes 122 (ten sets of three electrodes each) and two ring electrodes 120 for a total of 32 electrodes 134.

Examples of leads with segmented electrodes include U.S. Patent Application Publications Nos. 2010/0268298; 2011/0005069; 2011/0078900; 2011/0135803; 2011/0135816; 2011/0135817; 2011/0135818; 2011/0078900; 2011/0238129; 2011/0363500; 2012/0016378; 2012/0046710; 2012/0071949; 2012/0165911; 2012/0197375; 2012/0203366; 2012/0203320; 2012/0203321; 2013/0197602; 2013/0261684; 2013/0325091; 2013/0367587; 2014/0039587; 2014/0353501; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. A lead may also include a tip electrode and examples of leads with tip electrodes include at least some of the previously cited references, as well as U.S. Patent Application Publications Nos. 2014/0296953 and 2014/0343647, all of which are incorporated herein by reference in their entireties. A lead with segmented electrodes may be a directional lead that can provide stimulation in a particular direction using the segmented electrodes.

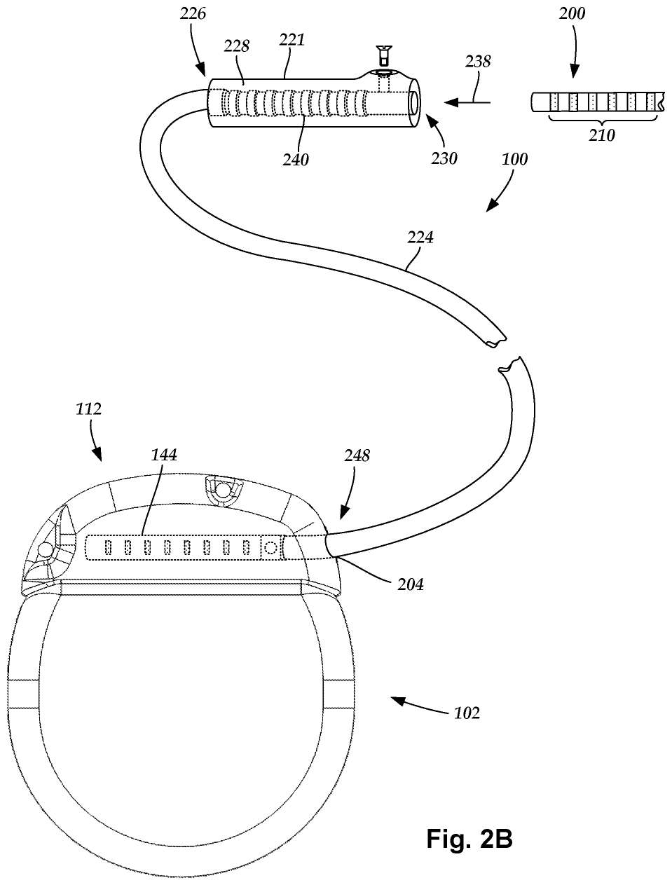

FIG. 3 is a schematic overview of one embodiment of components of an electrical stimulation system 350 including an electronic subassembly 360 disposed within an implantable pulse generator. It will be understood that the electrical stimulation system can include more, fewer, or different components and can have a variety of different configurations including those configurations disclosed in the stimulator references cited herein.

Some of the components (for example, power source 362, antenna 368, receiver 352, processor 354, and memory 355) of the electrical stimulation system can be positioned on one or more circuit boards or similar carriers within a sealed housing of an implantable pulse generator, if desired. Any power source 362 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the optional antenna 368 or a secondary antenna. The external power source can be in a device that is mounted on the skin of the user or in a unit that is provided near the user on a permanent or periodic basis.

If the power source 362 is a rechargeable battery, the battery may be recharged using the optional antenna 368, if desired. Power can be provided to the battery for recharging by inductively coupling the battery through the antenna to a recharging unit 366 external to the user. Examples of such arrangements can be found in the references identified above.

In one embodiment, electrical current is emitted by the electrodes 134 on the lead body to stimulate nerve fibers, muscle fibers, or other body tissues near the electrical stimulation system. A processor 354 is generally included to control the timing and electrical characteristics of the electrical stimulation system. For example, the processor 354 can, if desired, control one or more of the timing, frequency, amplitude, width, and waveform of the pulses. In addition, the processor 354 can select which electrodes can be used to provide stimulation, if desired. In some embodiments, the processor 354 may select which electrode(s) are cathodes and which electrode(s) are anodes. In some embodiments, the processor 354 may be used to identify which electrodes provide the most useful stimulation of the desired tissue.

Any processor 354 can be used and can be as simple as an electronic device that, for example, produces pulses at a regular interval or the processor can be capable of receiving and interpreting instructions from an external programming unit 358 that, for example, allows modification of pulse characteristics. In the illustrated embodiment, the processor 354 is coupled to a receiver 352 which, in turn, is coupled to the optional antenna 368. This allows the processor 354 to receive instructions from an external source to, for example, direct the pulse characteristics and the selection of electrodes, if desired. Any suitable memory 355 can be used and can store instructions, programs, algorithms, measurements, or the like or any combination thereof.

In one embodiment, the antenna 368 is capable of receiving signals (e.g., RF signals) from an external telemetry unit 356 which is programmed by a programming unit 358. The programming unit 358 can be external to, or part of, the telemetry unit 356. The telemetry unit 356 can be a device that is worn on the skin of the user or can be carried by the user and can have a form similar to a pager, cellular phone, or remote control, if desired. As another alternative, the telemetry unit 356 may not be worn or carried by the user but may only be available at a home station or at a clinician's office. The programming unit 358 can be any unit that can provide information to the telemetry unit 356 for transmission to the electrical stimulation system 350. The programming unit 358 can be part of the telemetry unit 356 or can provide signals or information to the telemetry unit 356 via a wireless or wired connection. One example of a suitable programming unit is a computer operated by the user or clinician to send signals to the telemetry unit 356.

The signals sent to the processor 354 via the antenna 368 and receiver 352 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 350 to cease operation, to start operation, to start charging the battery, or to stop charging the battery. In other embodiments, the stimulation system does not include an antenna 368 or receiver 352 and the processor 354 operates as programmed.

Optionally, the electrical stimulation system 350 may include a transmitter (not shown) coupled to the processor 354 and the antenna 368 for transmitting signals back to the telemetry unit 356 or another unit capable of receiving the signals. For example, the electrical stimulation system 350 may transmit signals indicating whether the electrical stimulation system 350 is operating properly or not or indicating when the battery needs to be charged or the level of charge remaining in the battery. The processor 354 may also be capable of transmitting information about the pulse characteristics so that a user or clinician can determine or verify the characteristics.

Another embodiment of an implantable pulse generator includes an IPG case, electronic subassembly disposed in the IPG case, two IPG connectors extending parallel to each other, and a power source (e.g., battery) disposed between the two IPG connectors.

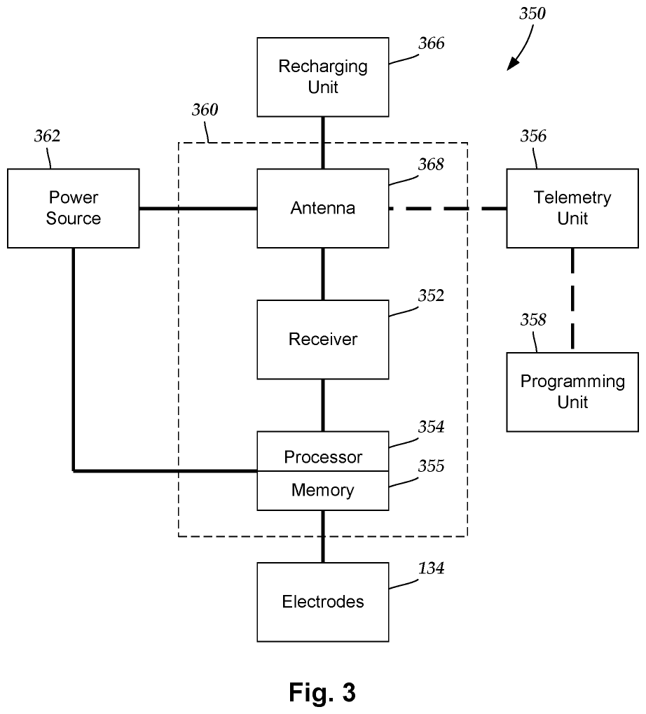

FIG. 4 is a cross-sectional view of a portion of one embodiment of an IPG 102 illustrating the IPG case 114 and electronic subassembly 110. The electronic subassembly 110 includes an integrated circuit device 405 which can include a processor (e.g., the processor 354 of FIG. 3) or any other suitable circuitry or any combination thereof. In at least some embodiments, the integrated circuit device 405 can be an ASIC (application-specific integrated circuit.) The electronic subassembly 110 may also include a printed circuit board 430 (PCB—FIG. 4) on which components, such as the integrated circuit device 405, are mounted.

It can be desirable to measure, observe, or record the temperature of an IPG case or tissue adjacent the IPG case. As an example, during charging the case temperature may vary by up to 2 degrees Celsius or more. Such a temperature measurement, observation, or reading can provide valuable information during manufacturing and storage, as well as providing continuous, periodic, or intermittent measurement, observation, or reading of tissue temperature when the IPG is implanted. For example, the temperature can be measured, observed, or recorded to obtain normal body temperature or to monitor the tissue or IPG case temperature during IPG operation or during charging of the power source of the IPG or during an MRI (magnetic resonance imaging) procedure which may generate eddy currents or other responses in the IPG. As another example, the temperature measurement can indicate when the IPG has been implanted as the temperature of the IPG case will change from room temperature to body temperature.

Some conventional IPGs include a thermistor that is attached to the case via wires, but this arrangement can complicate the manufacturing of the IPG due to the attachment to the case. As an example, a bead thermistor can be glued to a single point on the case with two wires that are attached to a printed circuit board (PCB) of the electronic subassembly. In addition to the manufacturing challenges, thermistor accuracy can be relatively poor (for example, +/−2 degrees Celsius).

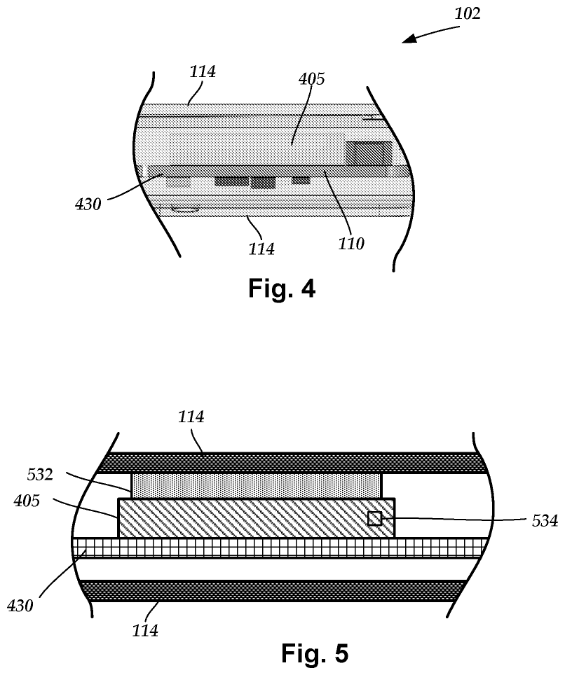

In contrast to these conventional IPGs, instead of employing a thermistor, a temperature sensor that is part of the integrated circuit device 405 (which is part of the electronic subassembly 110) can be used to monitor the temperature on or near the IPG case 114. FIG. 5 illustrates in cross-section a portion of an IPG 102 with the case 114, the integrated circuit device 405, a printed circuit board 430 (or other substrate) upon which the integrated circuit device is mounted, and a thermal coupling medium 532. The integrated circuit device 405 (such as an ASIC or other integrated circuit) of the IPG 102 includes an integrated temperature sensor 534 on board the integrated circuit device. Examples of such integrated temperature sensors are provided in U.S. Pat. No. 9,958,339, incorporated herein by reference in its entirety. The integrated temperature sensor 534 is often used to monitor the temperature of the integrated circuit device 405.

The thermal coupling medium 532 is positioned between, and in thermal contact with, the integrated circuit device 405 and the case 114 so that the temperature sensor 534 in the integrated circuit device is in thermal communication with the case in order to measure the temperature of the case or the tissue adjacent the case. In at least some embodiments, the thermal coupling medium 532 includes a solid, liquid, gel, or any combination thereof (for example, a liquid or gel disposed in a solid packaging). In at least some embodiments, the thermal coupling medium 532 is, or includes, any suitable thermal adhesive (e.g., a thermal epoxy), thermal tape, (for example, thermal adhesive disposed on a substrate), thermal pad, thermal paste, thermal grease, or the like or any combination thereof. The thermal coupling medium 532 has a thermal conductivity larger than the thermal conductivity of air and, preferably, at least 5, 10, 20, 50, 100, or more times the thermal conductivity of air. In at least some embodiments, the thermal coupling medium 532 is not electrically conductive or is a dielectric material. In at least some embodiments, the thermal coupling medium 532 is biocompatible or otherwise suitable for implantation over an extended period of time.

In at least some embodiments, the IPG case 114 is made of titanium or other metal and the integrated circuit device 405 is provided in a BGA (ball grid array) package. In at least some embodiments, the combination of BGA and titanium case, makes it easy to locate the top of the integrated circuit device 405 relatively close to the case 114. Other arrangements, materials, or packages of the integrated circuit device 405 and case 114 can also be used.

In at least some embodiments, the thermal coupling medium 532 is disposed on top of the integrated circuit device 405 opposite the printed circuit board 430 in a vertically stacked arrangement as illustrated in FIG. 5. In at least some embodiments, this arrangement with a thermal coupling medium 532 and a temperature sensor 534 in the integrated circuit device 405 has one or more of the following advantages over a thermistor: 1) simpler manufacturing; 2) better thermal performance; or 3) more accurate measurement.

In at least some embodiments, the integrated circuit device 405 or the IPG 102 can include a memory where temperature measurements can be stored. In some embodiments, the temperature measurements may be useful to determine when the IPG 102 was implanted (for example, when the temperature measurements change from room temperature to body temperature) or to monitor tissue/case temperature during charging, operation, or an MRI procedure. In at least some embodiments, the processor 354 (FIG. 3) may monitor the temperature and halt operation or charging if the temperature exceeds a threshold value or may transmit a warning message to the programming unit 358 or other external device.

An IPG 102 has been utilized as an example, but it will be understood that a thermal coupling medium can be used in any other implantable medical device having a case and an integrated circuit device with a temperature sensor. The thermal coupling medium is disposed between, and in contact with, the case and the integrated circuit device of the implantable medical device.

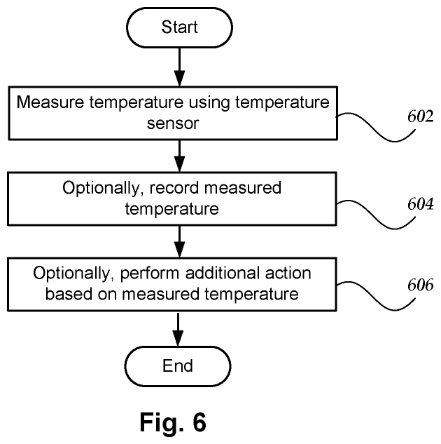

FIG. 6 illustrates one embodiment of a method of measuring the temperature of, or near, the case of the IPG (or other implantable medical device). In step 602, the temperature is measured using the temperature sensor of the integrated circuit device within the case of the IPG (or other implantable medical device). In step 604, the measured temperature is recorded. In step 606, one or more additional actions are optionally performed such as, but not limited to, determining whether the IPG (or other implantable medical device) is implanted, sending a warning if the measured temperature exceeds a threshold, halting or modifying operation of the IPG (or other implantable medical device) if the measured temperature exceeds a threshold, or the like or any combination thereof.

The above specification provides a description of the manufacture and use of the invention. Since many embodiments of the invention can be made without departing from the spirit and scope of the invention, the invention also resides in the claims hereinafter appended.

1.-15. (canceled)

16. An implantable medical device, comprising:
a case;
an integrated circuit device disposed within the case, the integrated circuit device comprising a temperature sensor; and
a thermal coupling medium disposed between, and in contact with, the case and the integrated circuit device, wherein the thermal coupling medium is a solid, liquid, gel, or any combination thereof.

17. The implantable medical device of claim 16, wherein the thermal coupling medium comprises a thermal adhesive, thermal tape, thermal paste, thermal grease, thermal pad, or any combination thereof.

18. The implantable medical device of claim 16, wherein the thermal coupling medium comprises a thermal epoxy.

19. The implantable medical device of claim 16, wherein the thermal coupling medium has a thermal conductivity that is at least five times a thermal conductivity of air.

20. The implantable medical device of claim 16, wherein the thermal coupling medium is not electrically conductive.

21. The implantable medical device of claim 16, further comprising a printed circuit board upon which the integrated circuit device is mounted.

22. The implantable medical device of claim 21, wherein the integrated circuit device is disposed between the printed circuit board and the thermal coupling medium.

23. The implantable medical device of claim 16, further comprising a power source disposed within the case.

24. The implantable medical device of claim 16, wherein the integrated circuit device comprises a processor configured for controlling operation of the implantable medical device.

25. The implantable medical device of claim 24, further comprising a memory coupled to the processor.

26. The implantable medical device of claim 25, wherein the memory comprises instructions, wherein the processor is configured to execute the instructions to perform actions including measuring a temperature using the temperature sensor.

27. The implantable medical device of claim 26, wherein the actions further include determining whether the implantable medical device has been implanted based on the measured temperature.

28. The implantable medical device of claim 26, wherein the actions further include recording the measured temperature in the memory.

29. The implantable medical device of claim 26, wherein the actions further include sending a warning when the measured temperature exceeds a threshold value.

30. The implantable medical device of claim 26, wherein the actions further include halting operation of the implantable medical device when the measured temperature exceeds a threshold value.

31. An electrical stimulation system, comprising:
the implantable medical device of claim 16; and
a stimulation lead configured for implantation into a patient and coupleable to the implantable medical device, the stimulation lead comprising
a lead body having a distal end portion and a proximal end portion,
a plurality of electrodes disposed at the distal end portion of the lead body,
a plurality of terminals disposed at the proximal end portion of the lead body, and
a plurality of conductive wires coupling the electrodes electrically to the terminals.

32. The electrical stimulation system of claim 31, further comprising a lead extension coupleable between the implantable medical device and the stimulation lead.

33. The electrical stimulation system of claim 31, wherein the thermal coupling medium comprises a thermal adhesive, thermal tape, thermal paste, thermal grease, thermal pad, or any combination thereof.

34. A method of measuring a temperature of, or near, a case of the implantable medical device of claim 16, the method comprising:
providing the implantable medical device; and
measuring a temperature using the temperature sensor.

35. The method of claim 34, further comprising sending a warning or halting operation of the implantable medical device if the measured temperature exceeds a threshold value.
